(19) United States
(12) Patent Application Publication
(10) Pub. No.: US 2025/0360002 A1
(43) Pub. Date: Nov. 27, 2025

(54) TISSUE GRAFT DELIVERY DEVICE

(71) Applicant: Graft Innovations LLC, Chandler, AZ (US)

(72) Inventor: Jorge Alberto Arjona, Chandler, AZ (US)

(21) Appl. No.: 18/869,239

(22) PCT Filed: May 24, 2024
(86) PCT No.: PCT/US2024/030920

§ 371 Date: Nov. 25, 2024

(51) Int. Cl. A61F 2/46

(52) U.S. Cl. CPC A61F 2/4603; A61F 2250/0058

(57) ABSTRACT

A tissue graft delivery system and associated devices are described. The tissue graft delivery devices utilized a conveyor belt moved by an actuator mechanism to shuttle a tissue graft, such as bone graft material, from a chute containing the graft into a surgical site, such as an intervertebral disc space. The interactive system and devices eliminate graft packing, inefficient tissue delivery, and other difficulties arising from friction between the bone graft material and graft contact surfaces of existing prior art systems, such as bone funnels.

### CROSS REFERENCE TO RELATED APPLICATIONS

This application claims priority of U.S. Provisional Application No. 63/544,511 titled “BONE GRAFT DELIVERY DEVICE” filed on Oct. 17, 2023, the disclosures of which are incorporated herein by reference in their entirety.

### BACKGROUND

### Technical Field

The disclosures herein relate to devices and methods of bone graft delivery. Specifically, the disclosures herein relate to a device and methods for quickly and precisely placing compositions containing bone graft material into a surgical site, such as an intervertebral disc space.

The spine functions to support the body, making walking and sitting upright possible. The 5 lumbar vertebrae in particular support about half the entire body weight when standing. The human spine consists of twenty-four (24) vertebral bones stacked one atop the other, including 7 cervical (neck), 12 thoracic, and 5 lumbar vertebrae. The cervical spine supports the head and allows for a wide range of motion of the head atop the neck. The thoracic vertebrae each articulates with two ribs (left and right) and provides axial support for the posterior (back) chest wall. It has the smallest range of flexion and rotation of the three vertebral regions. The majority of flexion and rotation of the body's trunk occurs in the lumbar region. The lumbar vertebrae are larger, thicker, and heavier than the thoracic and cervical vertebrae and are, consequently, able to support the larger forces arising from standing, walking, running, and more complex movements generated in this lowermost region of the spine. Because the lumbar vertebrae support so much of the body's weight and are subject to other forces, they are more susceptible to degenerative changes over a person's lifetime than other areas of the spine. The most common causes are trauma and chronic degenerative disease such as arthritis.

Each vertebral bone (vertebra) includes a vertebral body, a pair of lamina and a corresponding pair of pedicles behind the vertebral body forming an arch through which the spinal cord passes, and four facets (articular processes)—one pair above and one pair below—which articulate with the corresponding facets of the adjoining vertebral bones above and below to form four facet joints for each vertebra. Each of the 24 vertebral bodies are separated from the adjacent bodies above and below by a gel-like intervertebral disc (IVD). Like a gel pad, the IVDs cushion the adjacent vertebral bodies and distribute axial loads up-and-down the spine

Intervertebral disc disease affects “almost everyone,” according to a 1999 published review. The condition is the most common cause of chronic low-back pain in adults worldwide and has many etiologic causes. The final common result, regardless of cause, involves loss of the IVD's ability to support the body weight above the level of the degenerated, traumatized, or otherwise failed IVD. Over time, this insufficient support disrupts force balancing throughout the spine, damaging other IVDs and vertebrae giving rise to “multilevel disease.” A variety of painful and potentially debilitating conditions—including back muscle strain, subluxation (bone malalignment) of adjoining vertebral bodies, osteophyte (bone spur) development, arthritis of the facet joints, and IVD herniation with spinal nerve root impingement (“herniated disc” and “pinched nerve”)—result, causing chronic pain and sometimes localized or regional paralysis.

The economic impact of DDD is staggering. It is calculated that approximately 150 million workdays are lost each year in the U.S. due to lower back pain, most of which arises from DDD. Treating DDD is a manner that is effective and economical is, consequently, of great importance to a productive society. The only effective treatment for relief of debilitating pain and progressive nerve damage caused by DDD is surgical.

Over the past 25 years, instrumentation and evolving minimally invasive techniques have simplified spinal surgery and, particularly, interbody fusion procedures. Fusion between adjacent vertebral bodies requires replacing the intervertebral disc with a bone graft material bridging the intervertebral space and immobilizing the adjoining vertebra to be fused to allow propagation of the bone graft material with growth of bone across the space. Placement of an adequate amount of bone graft material to fill the intervertebral space is, therefore, important and bears directly on whether fusion ultimately occurs.

Surgical treatment of DDD typically removes the diseased IVD to alleviate chronic pain and allow the patient to become more active. IVD removal destabilizes the spine, and a means of restoring spinal stability and preserving the height of the intervertebral space is needed for patients undergoing this procedure. Although artificial IVD replacement devices exist, fusion of the adjoining vertebral bodies is the currently established treatment. A “spacer,” which can be fashioned from the patient's hip bone or manufactured from a thermoplastic or titanium, is typically used in spinal fusion surgery to maintain separation and assist with preserving the orientation of the vertebral bodies adjoining the intervertebral space. The spacer maintains the intervertebral distance, supports the weight of the body above that IVD level, may help preserve the normal curve of the spine (i.e., lordosis), and provides a frame to retain bone graft material placed by the surgeon to facilitate growth of bone into the intervertebral space and fusion of the vertebral bodies above and below the spacer. Bone graft material is also used to completely fill the intervertebral space around the spacer such that the entire IVD surfaces of the two adjoining vertebrae may fuse together via the bone graft material.

Transforaminal interbody lumbar fusion (TLIF) is one commonly used approach to interbody fusion surgery where the intervertebral space is accessed dorsolaterally (from the back/side of the spine) by removing the articular processes forming a facet joint from one side of the two adjacent vertebral bodies. This creates a portal through which the surgeon can safely access the intervertebral space via a small incision on the patient's back. Because the vertebral bodies are located centrally in the body, the distance from the small skin incision and the center of the 8-10-millimeter wide lumbar IVD space can be 4-6 inches in a non-obese adult. This distance can be much greater in the typical obese patient requiring interbody fusion surgery. There are other surgical approaches to access the intervertebral space in addition to the TLIF approach and all of these include accessing the intervertebral space a substantial distance from the skin incision. These techniques can be likened to building a model ship inside a bottle through the narrow neck of the bottle.

“Bone graft material” refers generally to a composition containing morcellized bone. The bone is often harvested from the patient's own iliac crest (hip bones) during the spinal fusion procedure (autologous bone autograft), ground into small spicules, and compressed into a gritty, pasty mass of ground bone with some fibrous connective bone. The bone graft material is then delivered into the intervertebral space following removal of the IVD. After partially packing the IVD space, a spacer can be positioned and then the remaining IVD space is packed with bone graft material to fill all the voids. Although placing and packing of bone can be done piecemeal by tamping small amounts of bone graft into the IVD space using forceps, this is a tedious process which can take thirty minutes or longer to complete. Surgeons can—and do—become impatient, which may result in leaving voids between the vertebral bodies within the IVD space which can compromise bone growth during healing which fuses the vertebral bodies. In a procedure that takes a total of 3-4 hours to perform, a substantial amount of time is spent simply packing bone graft material into the intervertebral space.

To address this issue, “bone funnels” were developed. A bone funnel is a funnel-shaped device with a long, straw-like stem used to access the cleared intervertebral space. This simplified accurate placement of bone graft material in the IVD space. Passage of the bone graft material through the stem, however, can be difficult and time consuming because the graft material is thick and viscous. Friction between the graft material and the bone funnel clogs the long, narrow stem. Devices inserted into the funnel to push graft material through the stem typically act only to further compress the graft within the stem without causing passage of graft through the device and into the IVD space. Also, because the graft material is axially loaded into the stem, graft material at the proximal end of the stem near the funnel is more densely packed than graft material distally at the opening of the stem from where the tissue graft is deposited at the surgical site. This can result in non-uniform density of tissue graft deposited at the surgical site. Wherein the tissue graft is bone graft deposited in the intervertebral disc space or into the paravertebral gutter, non-uniform graft density may hypothetically lead to non-uniform fusion.

Despite the long-term use of these currently available methods, they are time consuming and frequently ineffective in filling the intervertebral space sufficient to promote the highest rate of fusion between adjoining vertebral bodies following discectomy. Additional operating room (OR) time increases healthcare costs and longer anesthesia times increase the patient's risk of experiencing untoward cardiorespiratory events, postoperative infections, and other complications. Incomplete interbody fusion can result in intervertebral motion, collapse of the intervertebral space, nerve root compression, and serious neurologic complications leading to pain, numbness, and muscle weakness or partial paralysis requiring additional surgical treatment.

Consequently, there is a need for improved bone graft delivery devices and methods for placing bone graft material into a surgical site, such as an intervertebral disc space, which quickly deliver bone graft material to a precise location, improve bone graft delivery performance, increase the accuracy of graft placement, improve completeness of graft placement, reduce OR time needed for a fusion surgery, decrease healthcare costs, and decrease the rate or postoperative complications. Devices and methods for rapid, uniform loading of a tissue graft delivery device to facilitate controlled deposition of tissue graft, such as a bone graft, of uniform density to promote uniform healing are also needed.

For at least these and other reasons, new means for delivery of bone graft material and other tissue grafts to a surgical site, such as the intervertebral disc space and surrounding areas, are needed.

### BRIEF SUMMARY

The disclosures herein provide tissue graft delivery devices, loading devices for tissue graft delivery devices, and methods of use. Example embodiments of a bone graft delivery device can be used to deliver a bone graft, such as a bone graft preparation, to a surgical site. Disclosed example embodiments of the graft delivery device utilize a conveyor belt system mounted within a body of the device to substantially reduce dynamic friction between the tissue graft and non-belt surfaces of the device to facilitate controlled graft delivery into a surgical site. The belt forms the floor of a chute-shaped structure within the body, wherein the chute is configured as a trough open along the top and having an open (distal) end for delivery of the tissue graft from the device into the surgical site. After loading with graft material, the chute is covered with a sleeve to constrain the tissue graft within the chute during use.

The conveyor belt system includes a conveyor belt. In some embodiments, the belt is coupled to a pusher block. The chute is filled with tissue graft, such as bone graft for example. The loaded tissue graft rests within the chute atop the conveyor belt and, in some embodiments, forward of the pusher block. The loaded chute is then inserted into a closed-tip sleeve open on both ends to constrain the tissue graft within the chute, preventing a portion of the tissue graft from spilling through the open top of the chute during operation of the delivery device. In some embodiments, a plunger is mounted or inserted into the first (proximal) end of the sleeve to contact the pusher block. In some embodiments, the plunger contacts the tissue graft directly. The user then positions a second

(distal) end of the sleeve through a surgical incision at the anatomic location targeted for placement of the tissue graft. In some embodiments, the belt is manually activated, such as by the surgeon exerting thumb pressure on the plunger. In some embodiments, the belt is non-manually activated. Whether manually or non-manually actuated, movement of the graft-bearing conveyor belt moves the tissue graft out through an opening in the distal end of the sleeve and deposits the tissue graft, such as a bone graft, at the desired anatomic location.

Because the graft material rest upon and moves with the conveyor belt, dynamic friction between the graft material and the walls of the chute is substantially reduced. In some embodiments, dynamic friction is reduced by a factor of between about 20% and about 50% or more versus prior art bone graft delivery devices wherein a bone graft material is forced through a metal tube or funnel. The degree of dynamic friction reduction between the tissue graft and delivery device surfaces contacting the tissue graft versus prior art devices depends on many factors, including dimensional factors affecting the surface area of the delivery device in contact with the tissue graft, the width of the belt, the length of the belt, the material composition of the side(s) of the chute, the surface finish of the belt and side(s) of the chute, and other factors affecting a dynamic coefficient of friction between the tissue graft and the chute sides, as will be appreciated by those of skill in the art. Moreover, static friction between the graft-contacting surface of the belt and the graft material is significantly greater than the dynamic friction between the graft material and side(s) of the chute, wherein the graft moves with the belt and movement of the belt causes movement of the tissue graft through the chute.

Repeated testing of several chute and sleeve designs incorporated into prototype devices has consistently demonstrated this reduced friction manifest by the ability to easily move graft material from within the graft delivery device to the surgical site with a minimal actuation force. Fully functional patient-ready prototypes were assembled from component parts formed using a 3-D printer and loaded with fresh (moist) ground cadaver bone having physical characteristics essentially identical to allograft material created intraoperatively during an interbody fixation procedure. In the tested embodiments having a plunger contacting a pusher block coupled to the conveyor belt, actuation of the plunger with minimal thumb pressure consistently resulted in smooth, immediate, and controlled delivery of the desired amount of graft material out of the distal end of the sleeve through an opening in the distal end of the device. Essentially no packing of the graft material within the assembled chute-sleeve has been observed.

A tissue graft delivery device loading tray is also disclosed herein. The loading tray is configured to receive the chute such that a tissue graft, such as a bone graft, for example, is uniformly loaded from the loading tray into the chute longitudinally and not axially, as in prior art devices. Longitudinal loading is faster and provides an essentially uniform density of tissue graft along the length of the loaded chute versus axially loading of closed-tube tissue delivery devices of the prior art. The combination of (1) a loaded tissue graft having a substantially uniform density; and (2) a conveyor belt delivery mechanism of the tissue graft from the delivery device without packing of the tissue graft allows for fast, uniform, and controlled deposition of tissue graft, such as bone graft, from the tissue graft delivery device not available in existing prior art devices.

Disclosed is a tissue graft delivery device comprising a body having at least one open end; and a belt mounted to the body, wherein a first friction between a tissue graft contacting the belt and the belt exceeds a second friction between the tissue graft and the body causing the tissue graft to move axially through the body towards the open end and to exit the body through the at least one open end in response to a movement of the belt.

In some embodiments, the tissue graft delivery device further comprises an actuator functionally coupled to the belt, wherein the actuator causes the movement of the belt. In some embodiments, the actuator is a rod contacting a pusher block coupled to the belt. In some embodiments, the actuator comprises a rack-and-pinion gear mechanism. In some embodiments, the actuator is manually powered. In some embodiments, the actuator is electrically powered. In some embodiments, the tissue graft comprises a bone tissue.

In some embodiments, the body comprises a chute having a substantially elongate shape formed by at least one side wall and a floor defining a partially enclosed channel, wherein the belt is disposed within the channel, and a loading cutout forming a longitudinal opening in the channel opposite the floor, and a sleeve having a length equal to or longer than the chute length; a proximal end; and a distal opening, wherein the sleeve is configured to removably receive the chute and to constrain the tissue graft within the chute. In some embodiments, an actuator is movably disposed through the proximal end.

In some embodiments, the tissue delivery device additionally comprises a pusher block. In some embodiments, the pusher block is fixedly coupled to the belt and wherein distal movement of the pusher block within the chute in response to the actuator causes the belt to rotate.

Disclosed is a tissue graft delivery device comprising a body having a substantially elongate shape, at least one side wall defining a chute that is partially enclosed, and a distal opening; a belt mounted within the chute, wherein the belt forms a floor of the chute; a loading cutout extending axially along a portion of the length of the chute configured such that a tissue graft is loaded into the chute through the loading cutout; and a sleeve having a first (proximal) opening and configured to receive the body, wherein the sleeve constrains the tissue graft loaded into the chute; and wherein the tissue is moved out of the tissue graft delivery device through the distal opening in the chute in response to a movement of the belt.

In some embodiments, the belt is positioned opposite the loading cutout and generally parallel to the loading cutout.

Disclosed is a loading tray for a tissue graft delivery device, comprising a base having a chute receiver dimensioned to receive a chute assembly of a graft delivery device, a sloping side positioned proximate to the chute assembly loaded into the receiver and configured to direct a tissue graft placed on the sloping side into the chute positioned within the chute receiver; and a tamp configured to compress the tissue graft into the chute.

In some embodiments, the loading tray further comprises a top, wherein the top is elongated, comprises two sloping sides bounding an opening having a length about equal to the length of the chute.

Bone graft delivery devices are disclosed as examples of a tissue graft delivery device. A variety of example embodiments illustrating bone graft delivery devices during an interbody spinal fusion procedure are described, however these are not intended to be limiting. Persons of skill in the art will recognize that the embodiments of the tissue graft delivery devices, loading trays, and methods of use disclosed herein may be used in other anatomic locations during other surgical procedures, and utilizing other tissue grafts beyond bone grafts and techniques used in intervertebral spinal fusion procedures.

### SUMMARY OF THE DRAWINGS

FIG. 1 is an illustration of a surgeon using a bone graft delivery device from the prior art;

FIG. 2A is a bottom-right perspective view of a bone graft delivery device;

FIG. 2B is a top-left perspective view of a bone graft delivery device;

FIG. 3A is a proximal end view of a bone graft delivery device;

FIG. 3B is a distal end view of a bone graft delivery device;

FIG. 4A is a left side view of a bone graft delivery device;

FIG. 4B is a top view of a bone graft delivery device;

FIG. 4C is a bottom view of a bone graft delivery device;

FIG. 5 is a partially exploded view of a bone graft delivery device;

FIG. 6 is an exploded view of a bone graft delivery device;

FIG. 7 is s a cutaway view of a bone graft delivery device;

FIG. 8 is an enlarged partial cutaway view of a bone graft delivery device loaded with a tissue graft;

FIG. 9A is a top-right perspective view of an alternative embodiment of a bone graft delivery device;

FIG. 9B is a bottom-left perspective view of an alternative embodiment of a bone graft delivery device;

FIG. 10A is a proximal end view of an alternative embodiment of a tissue graft delivery device

FIG. 10B is a distal end view of an alternative embodiment of a tissue graft delivery device;

FIG. 11A is a left side view of an alternative embodiment of a tissue graft delivery device;

FIG. 11B is a top view of an alternative embodiment of a tissue graft delivery device;

FIG. 11C is a bottom view of an alternative embodiment of a tissue graft delivery device;

FIG. 12 is an exploded view of a body of an alternative embodiment of a tissue graft delivery device;

FIG. 13 is an exploded view of an alternative embodiment of a tissue graft delivery device;

FIG. 14 is a cutaway view of an alternative embodiment of a tissue graft delivery device;

FIG. 15A is a top perspective view of a loading tray for a tissue graft delivery device containing a chute of a tissue graft delivery device;

FIG. 15B is a bottom perspective view of a loading tray for a tissue graft delivery device;

FIG. 16A is a distal end view of a loading tray for a tissue graft delivery device;

FIG. 16B is a proximal end view of a loading tray for a tissue graft delivery device;

FIG. 17 is an exploded view of a loading tray for a tissue graft delivery device;

FIG. 18A is a side view of a loading tray for a tissue graft delivery device containing a chute of a tissue graft delivery device;

FIG. 18B is a top view of a loading tray for a tissue graft delivery device containing a chute of a tissue graft delivery device;

FIG. 18C is a bottom view of a loading tray for a tissue graft delivery device;

FIG. 19 is an exploded view of a loading tray for a tissue graft delivery device, including a chute of a tissue graft delivery device;

FIG. 20A is a perspective cutaway view of a loading tray for a tissue graft delivery device containing a chute of a tissue graft delivery device;

FIG. 20B is a side cutaway view of a loading tray for a tissue graft delivery device containing a chute of a tissue graft delivery device;

FIG. 21 is an exploded view of a loading tray for a tissue graft delivery device, combined with a exploded view of a chute of a tissue graft delivery device;

FIG. 22 is a diagrammatic representation of a method of using a tissue graft delivery device; and

FIG. 23 is a diagrammatic representation of a method of loading a tissue graft delivery device.

### DETAILED DESCRIPTION

Example embodiments of the devices and methods disclosed herein will now be presented with reference to the several drawing figures.

### Definitions

As used herein, “bone graft material” means any composition comprising bone in the form of fragments, ground bone, and demineralized bone compositions such as bone paste, putty, or gel. The bone may be living autologous bone autograft, decellularized bone matrix such as cadaver bone, or the like. When bone graft material is used in the context of an interbody spinal fusion procedure, bone graft material is typically a coarsely ground slurry of living autologous bone which is harvested from the patient during the interbody fusion procedure. Most commonly, the autologous bone is taken from a vertebral spinous process, a vertebral lamina, a vertebral facet, or the like. Less commonly, the autologous bone may be taken from a separate surgical site, such as the iliac crest. The bone, whether autologous graft, processed cadaver bone, or other source of bone graft known in the art is processed in the operating room by grinding the bone to form the slurry. Additional materials may be added to the slurry according to the surgeon's preference, such as biologic including growth factors, thickening agents, demineralized bone matrix with sodium hyaluronate (“bone putty”), other carrier materials used in the art, or the like.

As used herein, “proximal” means nearer to the user or further from the patient. “Proximal” means disposed more proximate to the user of a bone graft delivery device than one or more other structures.

As used herein, “distal” means away from the user; i.e., nearer to the patient, with respect to a bone graft delivery device or its individual components. The terms “distal” and “proximal” are used herein to relate the positions of structures with respect to one another using the user as a common reference point. The structures may include a bone graft delivery device or its individual components. For example, “proximal” means a structure or reference point of a bone graft delivery device which is disposed further from the patient (closer to the user) than a more “distal” structure of the bone graft delivery device. The relationship assumes the device is in use, however, the relative positions of the user and the patient are assumed regardless of whether a user or a patient are present.

As used herein, “axial” or “axially” refers to a directly parallel to a central longitudinal axis of a bone graft delivery device or other structure being referenced.

As used herein, directional references with respect to any of the several drawing figures, such as top, bottom, left, right, front, rear, upper, lower, and the like, for example, are intended for convenience of description to add clarity with reference to the object, region, or element discussed and is not intended to limit present disclosure or a component to a particular positional or spatial orientation.

As used herein, “radial” or “transverse” refers to a direction orthogonal to a central longitudinal axis of a structure.

As used herein, “circumferential” or “circumferentially” refers to a curved path around the body of a structure or sub-structure in a plane orthogonal to a central longitudinal axis.

As used herein, “additional embodiment,” “another additional embodiment,” “yet another additional embodiment,” “separate additional embodiment,” and similar terms refer to different examples of embodiments of bone graft delivery devices and related devices and methods disclosed herein within the scope of the disclosures and teachings found herein and the components thereof.

Attention will now be directed to providing detailed descriptions of several example embodiments of a bone graft delivery device, related devices, and methods of use with reference to the several drawing figures.

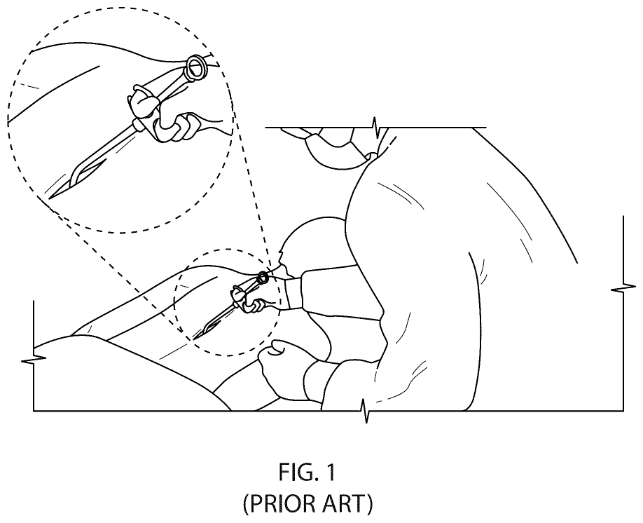

FIG. 1 is an illustration of a surgeon using a bone graft delivery device from the prior art. FIG. 1 shows a prior art bone graft delivery device in use by a surgeon. Wherein the operation is an interbody spinal fusion procedure performed on the lumbar spine, the patient may be positioned prone for a dorsal (through the back) incision. The bone used for grafting is typically living autologous bone harvested by the surgeon from a remote patient site, typically the iliac crest (hip bone). The harvested bone is ground into a coarse paste and then loaded into a prior-art bone delivery device, such as the bone funnel device shown in FIG. 1. A bone funnel is a generally tubular device with an elongated stem topped by a frustrum into which is loaded the ground bone paste. The surgeon then positions a distal end of the funnel stem through the incision to the desired site of implantation, such as the intervertebral space following removal of material forming the intervertebral disc. A plunger is used to force the bone paste from the frustrum out through the distal open end of the stem into the surgical site. As noted herein, movement of the bone paste is impeded by friction with the walls of the stem and becomes progressively more compacted by the plunger as it moves through the stem. As the bone paste becomes more compacted, resistant to the plunger increases and becomes difficult to overcome. Applying an increasing amount of force is time consuming and often requires striking the proximal end of a metal plunger with a hammer to force the compacted bone graft material through the distal open end of the stem.

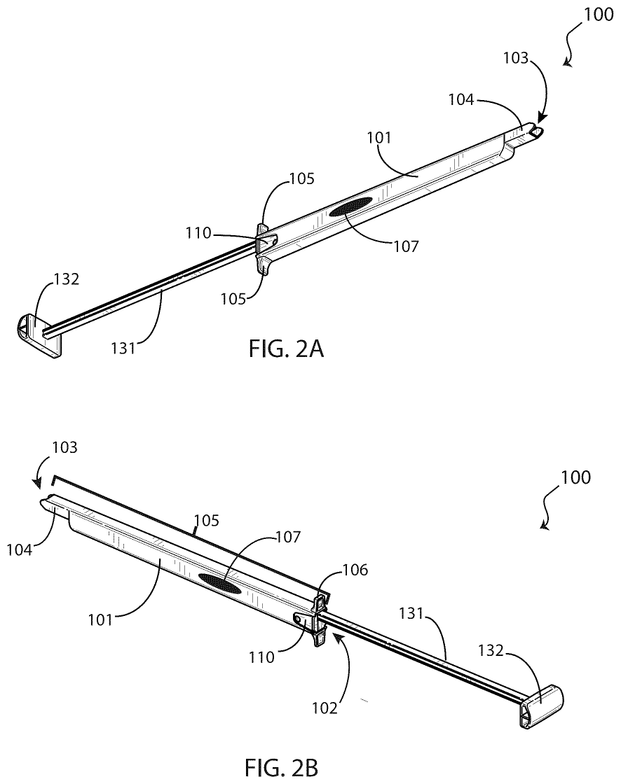

FIG. 2A is a bottom-right perspective view of a bone graft delivery device. FIG. 2B is a top-left perspective view of a bone graft delivery device. FIGS. 2A-B show a bone graft delivery device 100. Device 100, in some embodiments, comprises a sleeve 101 having a first (proximal) opening 102 configured to receive a body 110. A sleeve length 105 is shown extending between first (proximal) opening and second (distal) opening 103.

Sleeve 101 is a generally elongated unitary body with at least one side forming an internal cavity and having at least two openings. In some embodiments, including the example embodiment shown in FIGS. 2A-B, sleeve 101 has a rectangular cross section. This is not meant to be limiting, however. In some embodiments (not shown), sleeve 101 has an elliptical, a circular, or a polygonal cross section. In some embodiments, sleeve 101 tapers distally to form a spout 104 having a second (distal) opening 103. A bone graft, such as a prepared autologous bone graft, for example, is extruded through distal opening 103 into the surgical site during operation of bone graft delivery device 100. A first (proximal) opening is disposed opposite distal opening 103 and is configured to reversibly receive body 110 into the internal cavity. Depending on the actuation mechanism used (discussed in detail herein below), sleeve 101 may also include a pair of finger flanges 106 similar to the finger flanges at the proximal end of a typical syringe-type device. In some embodiments, sleeve 101 includes a grip feature 107 comprising ridges, bumps, surface roughening, or similar surface feature(s) to facilitate securely gripping sleeve 101 during use. In some embodiments, sleeve 101 and its listed constituent elements are formed as a unitary body. In some embodiments, grip feature 107 is an appliqué.

FIGS. 2A-B also show body 110 fully inserted through first (proximal) opening 102 of sleeve 101. Device 100 is configured such that body 110 may be removed and re-inserted into sleeve 101, in some embodiments. Sleeve 101 acts to constrain a bone graft within an elongate chute formed by body 110 for delivery through distal opening 103 in response to movement of the conveyor belt. Sleeve 101 also provides surfaces and features for the user to grip and manipulate device 100 while positioning distal opening 103 within the surgical site and activating the actuation mechanism; i.e., pushing a thumb pad 132, for example. The individual elements and mechanisms of body 110 are discussed in detail herein below.

Also shown are a rod 131 coupled to an actuator (not shown) and thumb pad 132 disposed on a proximal end of rod 131. In some embodiments of delivery device 100 having a manual actuation mechanism, rod 131 transmits a force applied to thumb pad 132 to a pusher block or other manual actuation mechanism to cause movement of a conveyor belt mounted within body 110.

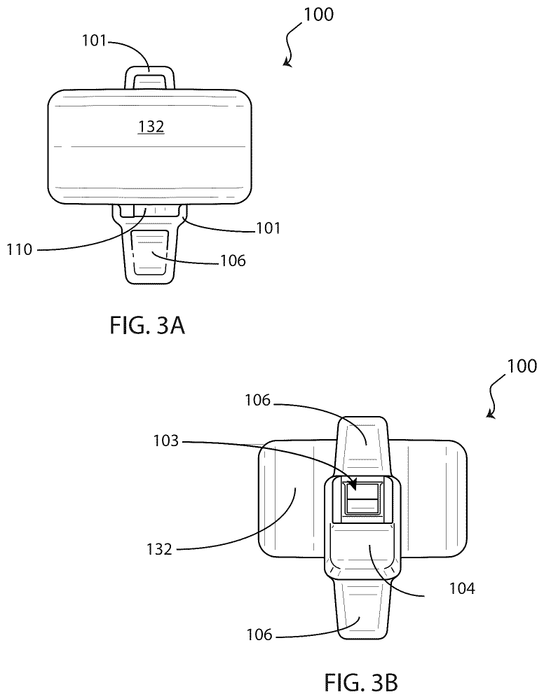

FIG. 3A is a proximal end view of a bone graft delivery device. FIG. 3B is a distal end view of a bone graft delivery device. FIGS. 3A-B show a proximal (rear) and distal (front) respectively end views of bone graft delivery device 100. Thumb pad 132 and a pair of finger flanges 106 are readily apparent along with the generally rectangular cross section of sleeve 101 and body 110. Spout 106 is shown in FIG. 3B leading into the internal cavity of sleeve 101 through second (distal) opening 103.

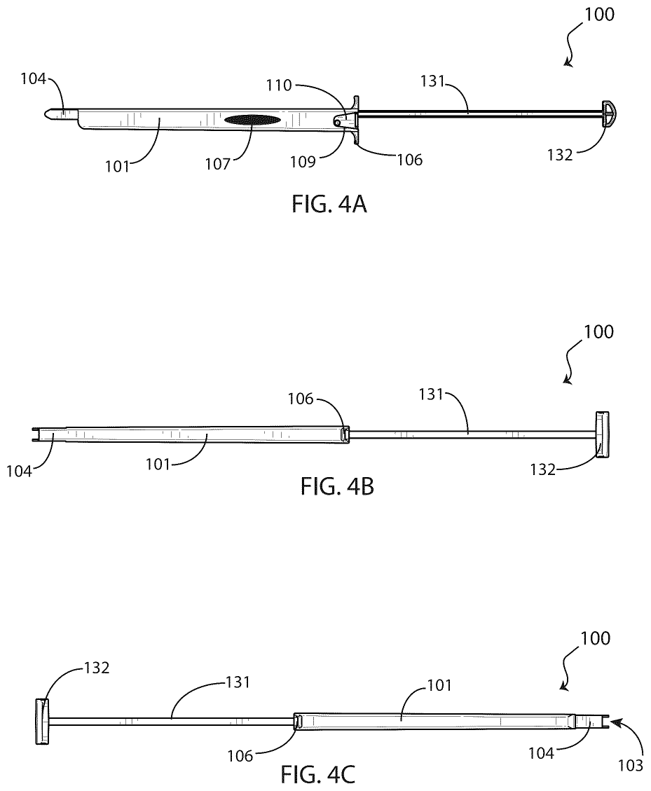

FIG. 4A is a left side view of a bone graft delivery device. FIG. 4B is a top view of a bone graft delivery device. FIG. 4C is a bottom view of a bone graft delivery device. FIGS. 4A-C show bone graft delivery device 100 from three additional viewpoints. Again, the substantially elongated form of sleeve 101 and device 100 generally is shown. The elongated form of device 100 has several advantages, including a sufficient length to insert second (distal) opening 103 deep within a surgical incision and to accommodate a relatively large volume of bone or other tissue graft material to save time by reducing the need to repeatedly load device 100 with additional bone graft material during a surgical procedure. FIG. 4A additionally shows a side cutout 109 within sleeve 101 through which body 110 can be grasped for removal from sleeve 101. Removal of body 110 from sleeve 101 is necessary, in these examples and some additional embodiments, for loading of a chute 111 with bone or other graft material, as will be subsequently discussed herein.

In some embodiments, the maximal outer dimensions of sleeve 101 measure about eight (8) millimeters high and about ten (10) millimeters wide. In some embodiments, the maximal outer dimensions of sleeve 101 measure about six (6) millimeters high and about eight (8) millimeters wide. In some embodiments, the maximal outer dimensions of sleeve 101 measure greater than about eight (8) millimeters high and about ten 10) millimeters wide.

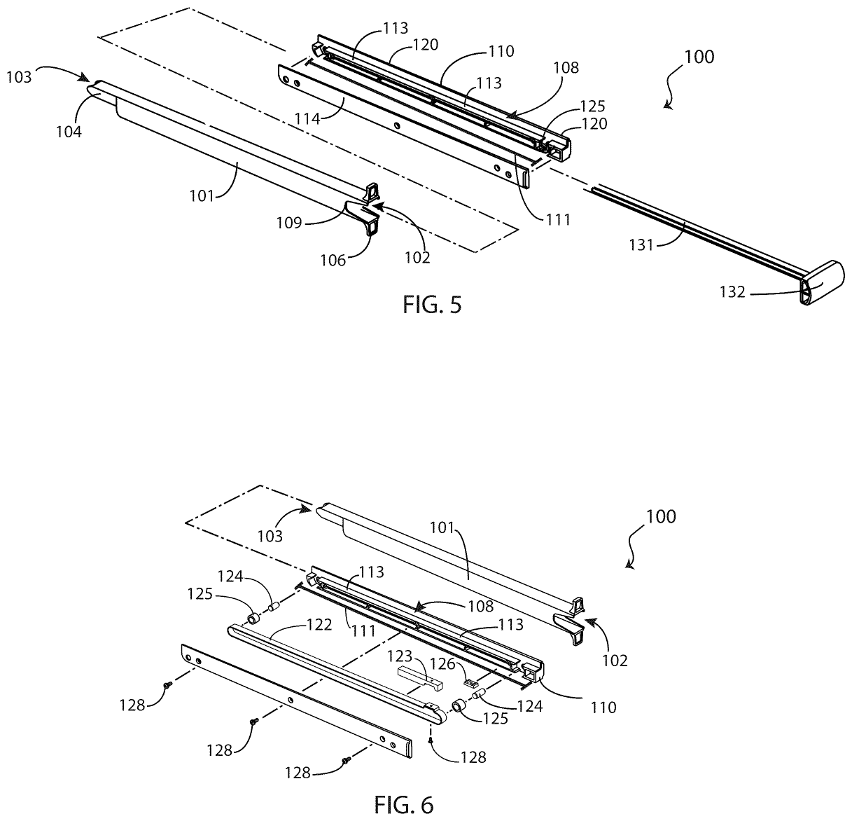

FIG. 5 is a partially exploded view of a bone graft delivery device. FIG. 54 shows delivery device 100 with body 110 detached from chute 111. Body 110 is partially exploded showing a closeout 114 and a sidewall 120 forming two sides of a graft chamber 115 in this example and in some embodiments. Body additionally houses belt 122 and various belt-related structures discussed further below. Belt 122, side wall 120, and closeout 114 define a three-sided chute 111 which is open at the top, in some embodiments. An open top 108 of chute 111 reduces total friction between bone graft material and the chute 111, as compared to a fully closed chute structure. Open top 108 also provides a means for loading bone graft material into chute 111 of delivery device 100. Body 110 with its constituent substructures is dimensionally configured to slide through proximal opening 102 into sleeve 101. Sleeve 101, as discussed herein above, provides a gripping feature 107, in some embodiments, and additionally constrains bone graft material within chute 111 for deposition at the surgical site through distal opening 103 of spout 104.

In some embodiments, chute 111 has a volume of about five 5 cubic centimeters (“cc”.) In some embodiments, chute 111 has a volume of about ten (10) cc. In some embodiments, chute 111 has a volume of between about ten (10) and about thirty (30) cc. In some embodiments, chute 111 has a volume of greater than about thirty (30) cc. In some embodiments, chute 111 has a volume of about three (3) cc.

Also shown in FIG. 5 are thumb pad 132 and rod 131 of the actuator mechanism.

FIG. 6 is an exploded view of a bone graft delivery device. FIG. 6 shows additional structures forming the actuator mechanism in this example and some embodiments. Belt 122 moves in response to the actuator mechanism of graft delivery device 100. In the examples discussed in detail herein, the actuator mechanism comprises a pusher block 123, rod 131, thumb pad 132. Pusher block 123 is coupled to belt 122 by a belt retainer 126 and a fastener 128. Rod 131 transmits a force on thumb pad 132 to pusher block 124. In a configuration wherein body 110 is fully inserted through proximal opening 102 into sleeve 101, rod 131 is inserted into a proximal end of body 110 to abut pusher block 123. Pressure transmitted onto pusher block 123 by a user through rod 131 causes pusher block 123 to move distally. Distal movement of pusher block 123 causes counterclockwise rotation of belt 122 when viewed from the left side of bone graft insertion device 100. Distal movement of pusher block 123 along with belt 122 shuttles bone graft material distally along chute 111 and out of distal opening 103. Belt 122 exerts a friction force against the bone graft material contacting belt 122 along a base of chute 111, urging the entire mass of graft material distally rather than simply pushing the graft material through a distal opening wherein friction with the sidewalls of a prior art device compresses the graft material rather than moving the graft material out of the device.

The use of a conveyor belt, such as belt 122, for example, to move bone or other tissue graft material from within a delivery device to a surgical site is a central feature of the example embodiments of the tissue graft delivery devices discussed herein. Although the belt is operationally coupled to a suitable actuator mechanism, specifics of the actuator mechanism are of lesser importance. A manually operated plunger actuator mechanism is shown in the several drawing figures, but other actuator mechanisms can be used to move a pusher block-like structure, or related structure coupled to belt 122. For example, in some embodiments, a manually operated trigger or lever coupled to a rack-and-pinion gear moves a pusher block to rotate belt 122. In some embodiments, a worm gear mechanism moves a pusher block to rotate belt 122. In some embodiments, an electric motor powered by a battery, such as a disposable or a rechargeable battery, and electrically coupled to a suitable prior art control switch mechanism moves the actuator mechanism (by rotating a worm gear, for example) to move a pusher block or otherwise rotate belt 122. An electric motor drive is particularly suited to drive the worm gear actuator mechanism, as would be appreciated by a person of skill in the art. In some embodiments, compressed air activates the actuator mechanism to move a pusher block to rotate belt 122.

FIG. 7 is s a cutaway view of a bone graft delivery device. FIG. 7 shows a cutaway view taken along a (central, median) long axis of bone graft delivery device 100. Chute 111 is defined by components of body 110 and belt 122 which are, in turn, inserted into sleeve 101. A belt movement 140 of rotating belt 122 causes a tissue graft loaded into chute 111 and thus contacting belt 122 to move axially through the body towards distal opening 117, and to exit the body through distal opening 117 in response to the actuator. Belt movement 140 is indicated by the labeled arrow in FIG. 7 Chute 111 has a general “U” shape with belt 122 forming the bottom and closeout 114 along with sidewall 120 forming the sides of the “U.” The top of chute 111 is open, in some embodiments, although when inserted into sleeve 101, as shown in FIG. 7, sleeve 101 covers the top of chute 111. When compared to a fully closed-in stem of a prior art bone graft delivery device, such as a bone funnel, friction in chute 111 is reduced by a factor of 50%-80%.

As shown by FIG. 7, there is a first friction acting in the direction of arrow 150 between an outer chute-contacting surface of the mass of bone graft material and closeout 114 along one side and sidewall 120 along the other side of chute 111. First friction 150 resists movement of the bone graft material distally through opening 103. First friction 150, however, is readily overcome by a second friction acting in the direction of arrow 151. Second friction 151 is created at the interface between an upper surface of belt 122 and an outer belt-contacting surface of the mass of bone graft material. Second friction 151 acts in a distal direction opposite first friction 150, which acts in a proximal direction. Further, although body 110 is enclosed within sleeve 101 during operation of delivery device 100, chute 111 remains “open” on top as the top aspect of sleeve 101 is configured to be slightly above chute 111 and only contacts stray bits of the bone graft material which may exude above the upper aspect of chute 111 during operation. In this manner, sleeve 101 constrains the bone graft material within chute 111 until the graft material reaches distal opening 103 of graft insertion device 100. Consequently, instead of a first friction exerted circumferentially by all walls bounding bone graft material contained in a prior art bone funnel stem or related prior art delivery device, first friction 150 resisting movement of the bone graft out of distal opening 103 is, at most, exerted against only about 20% to about 60% of the collective outer surface of bone graft material contained within chute 111 of graft insertion device 100. This fractional reduction in proximal-acting friction essentially prevents any packing of the bone graft material and facilitates rapid extrusion of the bone graft through distal opening 103 into the desired patient anatomic site, such as an intervertebral space or paraspinous gutter, for example.

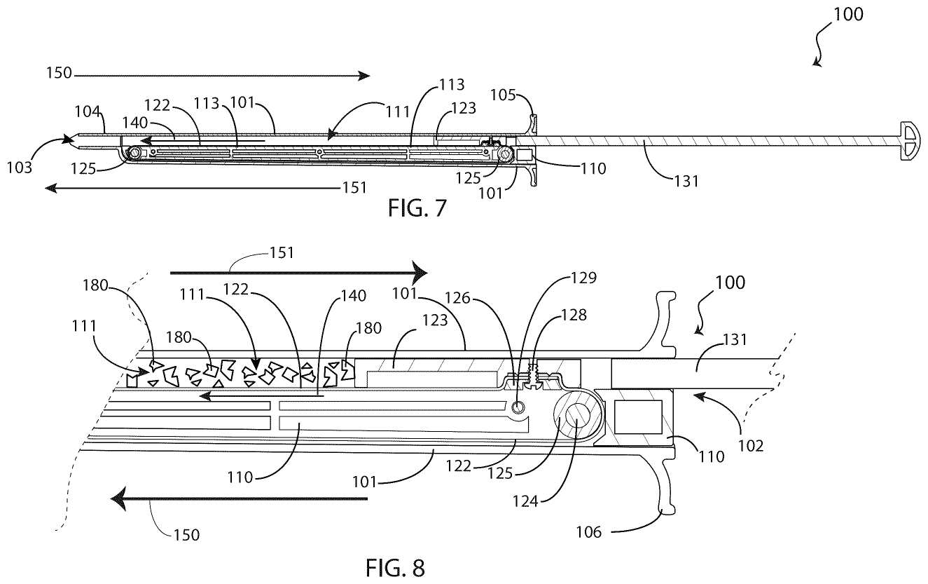

FIG. 8 is an enlarged partial cutaway view of a bone graft delivery device loaded with a tissue graft. FIG. 8 shows details of a mounting system for belt 122 within body 110 of bone graft delivery device 100. Belt 122 is mounted to body 110, in some embodiments, through a system of pins, bushings, and belt supports. For example, in some embodiments, belt 122 passes over and is partially suspended between at least one bushing 125. Each bushing 125 is mounted to body 110, such as on a pin 124. In some embodiments, floor 113, as shown in FIG. 6 and FIG. 7, e.g., runs beneath belt 122 along chute 111 and supports the weight of a tissue graft 180, such as bone graft material, e.g., resting on belt 122 within chute 111. Belt movement 140 is shown by the labeled arrow in FIG. 8.

In some embodiments, belt 122 is a continuous band of material formed into a belt. In some other embodiments, including the example embodiments discussed herein and shown in the several drawing figures, belt 122 is formed by coupling two ends of a narrow strip of a belt-forming material with a belt retainer 126. In the example embodiments shown in FIG. 8, belt retainer 126 is a plate having a central hole through which passes a fastener 128, such as a screw, into pusher block 123. Fastener 128 passes through belt retainer 126, through each of two ends of belt 122, and is received by a hole disposed in pusher block 123. In this example manner, belt 122 is securely fixed to pusher block 123 such that any linear movement of pusher block 123 translates into rotation of belt 122. Pusher block 123 is coupled to belt 122 by fastener 128, and block 123 therefore cannot slip on belt 122 so long as belt 122 remains intact and does not tear or rupture, in some embodiments of device 100 comprising pusher block 123.

In some alternative embodiments (not shown), belt is formed as a continuous loop of the belt-forming material. In some embodiments, belt 122 formed from the continuous loop of the belt-forming material is screwed or otherwise fastened to pusher block 123. In some embodiments, a clip, pin, brad, adhesive, or other configuration of fastener 128 is used to coupled belt 122 to pusher block 123. In some embodiments, pusher block 123 is fixedly coupled to rod 131. In some embodiments, pusher block 123 is fixedly coupled to an alternative actuator mechanism, such as one of the gear mechanisms discussed herein above, for example. In some embodiments, pusher block 123 is reversibly coupled to an alternative actuator mechanism.

The actuator mechanism acts upon pusher block 123, in some embodiments and as described throughout these disclosures. As shown in FIG. 8, rod 131 of the manual plunger-type actuator mechanism is brought into contact with pusher block 123 through proximal opening 102 of sleeve 101, after which additional movement of rod 131 in an axially inward direction through opening 102 moves pusher block distally through chute 111 causing belt 122 to rotate. The combination of distal movement of pusher block 123 against the bone graft material contained within chute 111 and second friction 151 substantially fixing graft 180 to belt 122 shuttle graft 180 distally through chute 111 and out through distal opening 103 of sleeve 101.

Belt 122 may be formed from a suitable biocompatible material, such as thermoplastic including polyvinyl chloride (“PVC”), polyurethane, and other suitable thermoplastic polymer materials. Belt 122 may also be formed from alternative materials having a biocompatible coating. In some embodiments, the surface of belt 122 is corrugated or presents similar surface features or biocompatible coatings that increase friction between the belt surface and the bone graft composition.

FIG. 8 also shows a fastener boss 129. In some embodiments, one or more fastener bosses 129 are formed into the material of body 110 to receive a corresponding number of fasteners 128, such as screw ore the like, used to secure closeout 114 to the remainder of body 110.

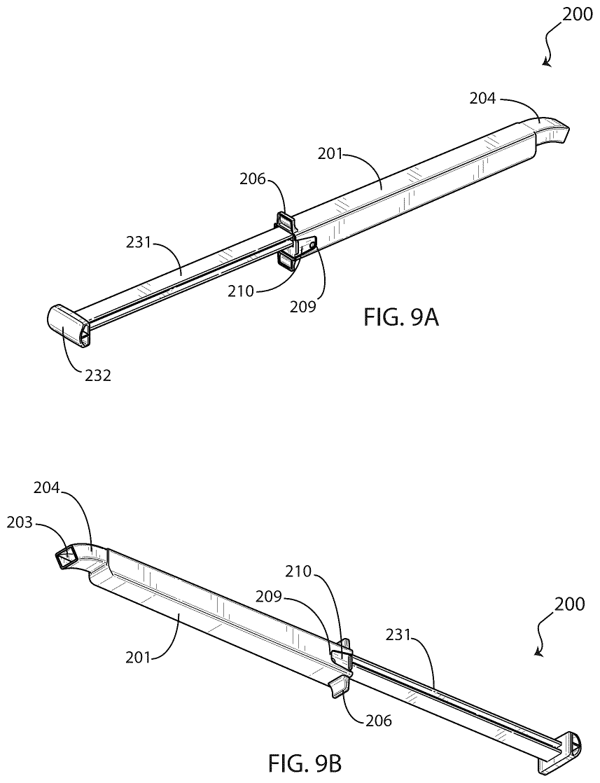

Alternative embodiments of a bone or tissue graft delivery device may be formed using shapes and dimensions to accommodate other uses, such as when loading of larger amounts of bone graft material is needed for deposition into surgical sites other than the intervertebral space. An example of such an embodiment is shown in FIGS. 9-15 and discussed below.

FIG. 9A is a top-right perspective view of an alternative embodiment of a tissue graft delivery device and FIG. 9B is a bottom-left perspective view of an alternative embodiment of a tissue graft delivery device. FIGS. 9A-B show a tissue graft delivery device 200. Device 200 comprises a sleeve 201 that receives a body 210. Sleeve 201 is a substantially elongated unitary body formed in the shape of a hollow tube and having a first (proximal) opening 202 and a second distal opening 203. In some embodiments, distal opening 203 is disposed on a spout 204. Spout 204 is shaped, as shown in FIGS. 9A-B, to direct a tissue graft, such as a bone graft material, into a precise anatomic surgical site for delivery of the tissue graft. In some embodiments, sleeve 201 additionally comprises a cutout 209 disposed at second (distal) opening 203 to facilitate insertion and removal of body 210 into and from sleeve 201. In some embodiments having a manual actuation means, such as a plunger (not shown) coupled to a rod 231, sleeve 201 additionally comprises a finger flange 206 to facilitate a user maintaining a stable grip on device 200 during activation of an actuation means, whether manual or powered, to extrude the tissue graft material out of second (distal) opening 203 into the desired anatomic location.

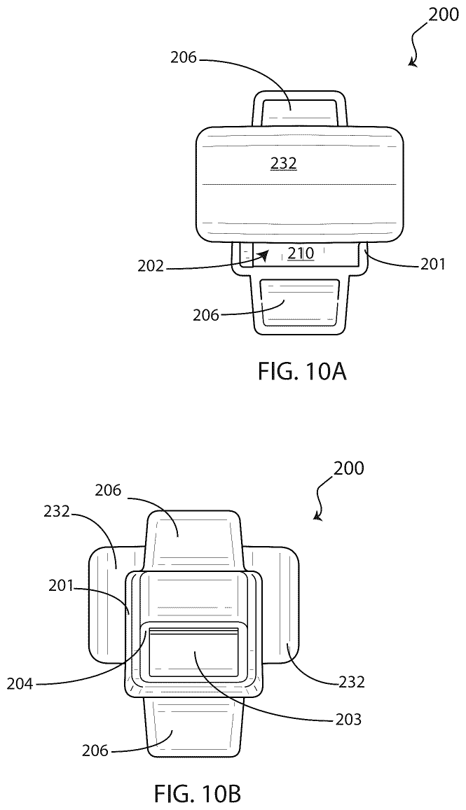

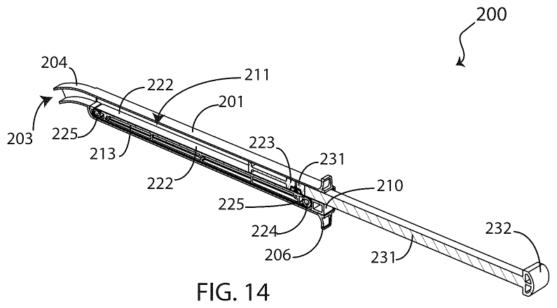

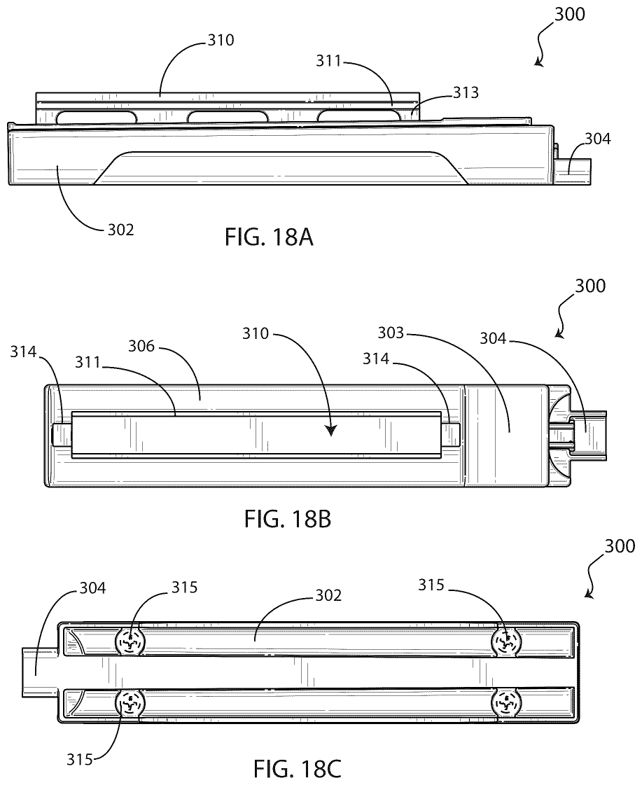

FIG. 10A is a proximal end view of an alternative embodiment of a tissue graft delivery device. FIG. 10B is a distal end view of an alternative embodiment of a tissue graft delivery device. FIGS. 10A-B show opposing end views of the alternative embodiment of the graft delivery device 200. Sleeve 201 receives body 210 through first (proximal) opening 202. Body 210 includes a chute 211 (see, e.g., FIG. 13, FIG. 14, and FIG. 18) loaded with a tissue graft, such as a bone graft, for example. After loading chute 211 with the tissue graft, body 210 is passed through first (proximal) opening 202 of sleeve 101 such that sleeve 101 encloses chute 211 to prevent spillage of the tissue graft from an open top 208 of chute 211 during graft delivery.

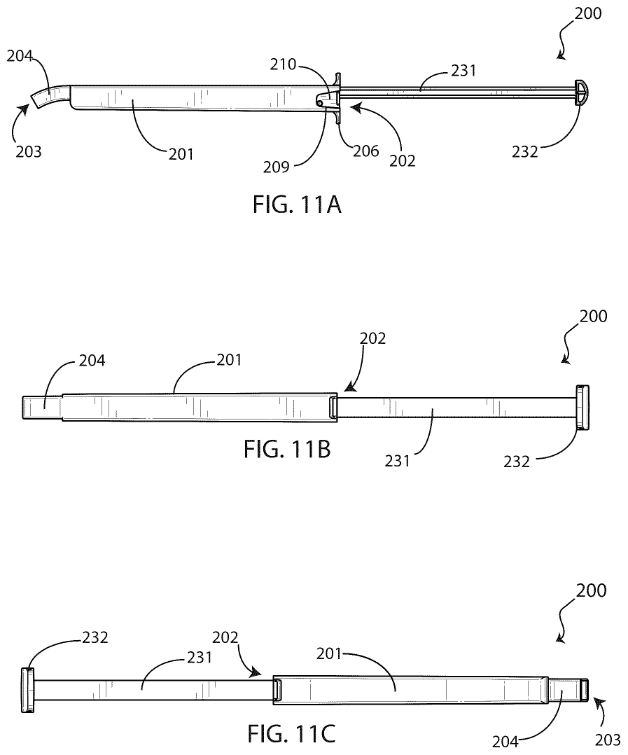

FIG. 11A is a left side view of an alternative embodiment of a tissue graft delivery device. FIG. 11B is a top view of an alternative embodiment of a tissue graft delivery device. FIG. 11C is a bottom view of an alternative embodiment of a tissue graft delivery device. FIGS. 11A-C show device 200 from the side, top, and bottom. The example embodiment of device 200 shown in FIGS. 11A-C is configured with a manual actuator comprising rod 231 and thumb pad 232. Rod interacts with pusher block 223, whereunder manual pressure on thumb pad 232 is transmitted axially down rod 231 to pusher block 223, moving pusher block 223 axially down chute 211 in a proximal to distal direction, causing belt 222 to move, shuttling tissue graft 280 axially through chute 211 and out of device 200 through spout 204 into the desired anatomic site. In some embodiments, side cutout 209 facilitates insertion and removal of body 210 from sleeve 201 without displacement or activation of an actuator means, particularly when chute 211 of body 210 is loaded with tissue graft 280 to prevent premature, inadvertent, unwanted spillage or expulsion of graft 280 from device 200.

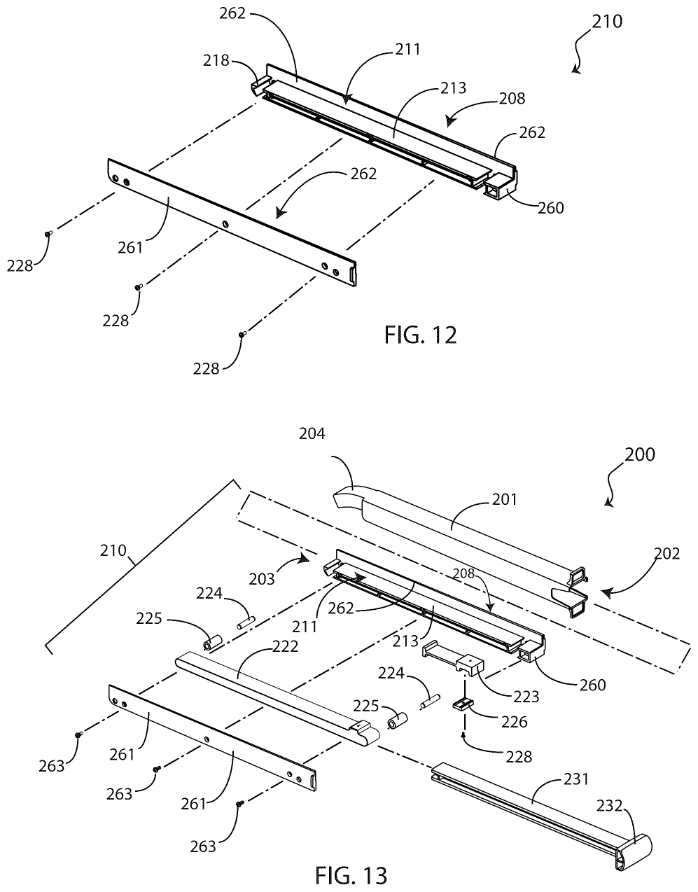

FIG. 12 is an exploded view of a body of an alternative embodiment of a tissue graft delivery device. FIG. 12 shows body 210 comprising a main body 260 and a side body 262 coupled together with a plurality of fasteners 228. In some embodiments, main body 260 and side body 262 are coupled together using alternative means, such as annealing, use of adhesives, or other means utilized to rigidly couple together structural elements of a medical device. Embodiments of body 210 of device 200 or body 110 of device 100 comprise substructure means to rotatably mount components allowing operation of belt 222. One example of such means is the pully system comprising one mor more bushings 225, each mounted on pin 224 disclosed herein, in some embodiments. This belt-mounting structure is by way of example only and not intended to be limiting. Other means to rotatably mount a conveyor belt within a structure, such as mounting belt 222 within body 210, such as those known to those of skill in the mechanical arts, are feasible and considered within the scope of the disclosures provided. Floor 213 and belt support 218, as shown in FIG. 12, are formed as part of body 210 and act to support belt 222, in some embodiments.

In some embodiments, body 210 is formed as a unitary body without fasteners 228 or other fastening means. Body 210 may be formed from a biocompatible, substantially rigid, medical-grade plastic or similar polymer. Some non-exclusive examples of such polymers include polypropylene, polycarbonate, polyethylene, acrylonitrile butadiene styrene, polymethyl methacrylate (acrylic), polyoxymethylene (polyacetal), and polyamide (nylon) and will be known to those of skill in the medical device arts. Manufacture of body 210 formed as a unitary body is accomplished, for example, by standard injection molding techniques for medical and other devices.

FIG. 13 is an exploded view of an alternative embodiment of a tissue graft delivery device. FIG. 13 shows device 200 comprising an exploded view of body 210 containing multiple sub components. In the example shown, 222 is mounted upon two bushings 225 each rotatably mounted upon a pin 225. Pins 225 are, in turn mounted upon a pin support boss or similar structure (not shown) coupled to or molded within main body 260 and/or side body 261 of body 210, in some embodiments. Pusher block 223 and belt retainer 226 are fixedly mounted to belt 222 by fastener 228, in this example and some embodiments. Thus, the belt mounting means are enclosed within body 210, as shown. An actuation means, such as rod 231, in some embodiments, is configured to interact with pusher block 223 to effect movement of belt 222.

Also shown in FIG. 12 and FIG. 13 is chute 211. Chute 211 is a space formed from main body 260 and side body 261 and is bounded by at least one side wall 262, floor 213 and an open top 208. Tissue graft 280, such as a material comprising bone graft, is loaded into chute 211 in preparation for delivery to the surgical site. After loading chute 211 with the desired type and quantity of tissue graft 280, body 260 and its assembled sub components, such as shown in FIG. 13, are passed through first (proximal) opening 202 into sleeve 201 whereupon device 200 is ready for use.

FIG. 14 is a cutaway view of an alternative embodiment of a tissue graft delivery device. FIG. 14 shows device 200 comprising body 210 inserted into sleeve 201. A mechanical interaction between rod 231 and pusher block 223 wherein rod 231 contacts pusher block 223. A manual pressure applied to thumb pad 232 is transmitted through rod 231 to pusher block 223. Pusher block 223 is coupled to belt 222, consequently movement of pusher block 223 in response to movement of rod 231 causes belt 222 to rotate. Static friction between belt 222 an tissue graft 280 (not shown in FIG. 14) allows belt 222 to propel graft 280 distally along chute 211, whereupon tissue graft 280 exits second (distal) opening 203 of sleeve 201 and is deposited at the user's desired surgical anatomic site. In the embodiment shown in FIG. 14, and in some other embodiments, pusher block 223 additional propels tissue graft 280 distally through chute 211 through pressure transmitted to graft 280 resting on belt 222 against pusher block 223.

A loading tray is provided for the various embodiments of tissue graft delivery devices disclosed herein. The loading tray is advantageous because it provides a means to stabilize the tissue graft delivery device for loading of a tissue graft, such as a bone graft. Elements of the loading tray include a base that receives a body of the graft delivery device, a top shaped to evenly deliver tissue graft into an open top of a trough of the graft delivery device, and a tamp configured to uniformly pack the loaded tissue graft to a desired density within the trough of the graft delivery device.

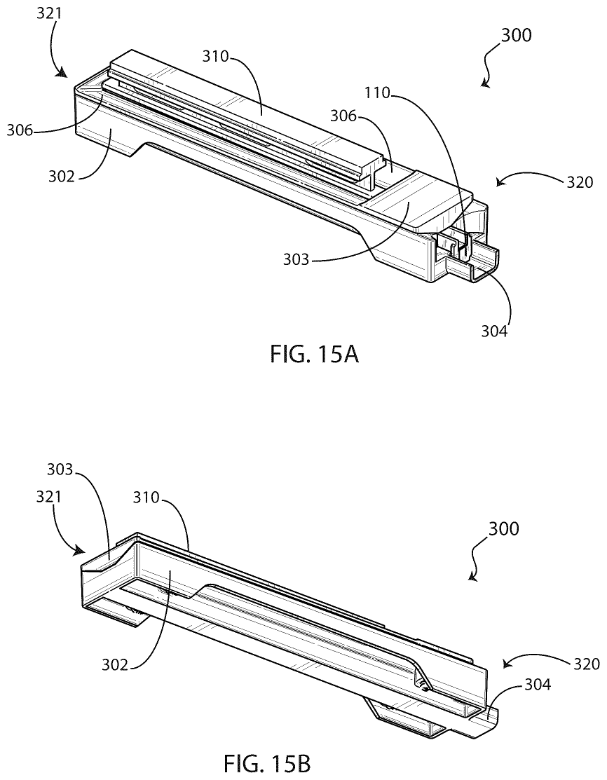

FIG. 15A is a top perspective view of a loading tray for a tissue graft delivery device containing a chute of a tissue graft delivery device and FIG. 15B is a bottom perspective view of a loading tray for a tissue graft delivery device. FIGS. 15A-B show a loading tray 300. Loading tray 300 comprises a base 302 configured to receive a body 210 of a tissue graft delivery device removably coupled to a top 303. A tamp 310 is used to urge a tissue graft, such as a bone-graft material, through an opening 207 in top 303 into a chute, such as chute 110 or chute 220, for example, of a tissue graft delivery device, such as device 100 or device 200, for example. Structures in the several drawing figures illustrating loading tray 300 are shown containing elements of device 100 or device 200 disclosed herein above, by way of example only, to demonstrate functional elements and methods of use of loading tray 300.

FIG. 15A shows body 210 of tissue graft delivery device 200 inserted into a receiver 303 of base 302. In some embodiments, receiver 303 is molded into a shape of base 302 and dimensioned to receive body 210. Also shown is top 303 configured with a pair of sloping sides 306 configured to direct the tissue graft material through opening 207 into chute 211 of the loaded graft delivery device. Tamp 310 is dimensioned, in some embodiments, to partially pass through opening 207 to facilitate urging the tissue graft material through opening 207 into chute 211 and to pack the loose tissue graft material evenly along the length of chute 211 in a gentle manner controlled directly by the user's hand manipulating tamp 310.

Although not meant to be limiting, formation of base 302, top 303, and tamp 310 are of separate unitary bodies. Established techniques, such as injection molding, 3-D printing, extrusion, and the like may be used to from components of loading tray 300 from biocompatible medical device-grade plastics, aluminum, stainless steel, or other alloys, e.g.

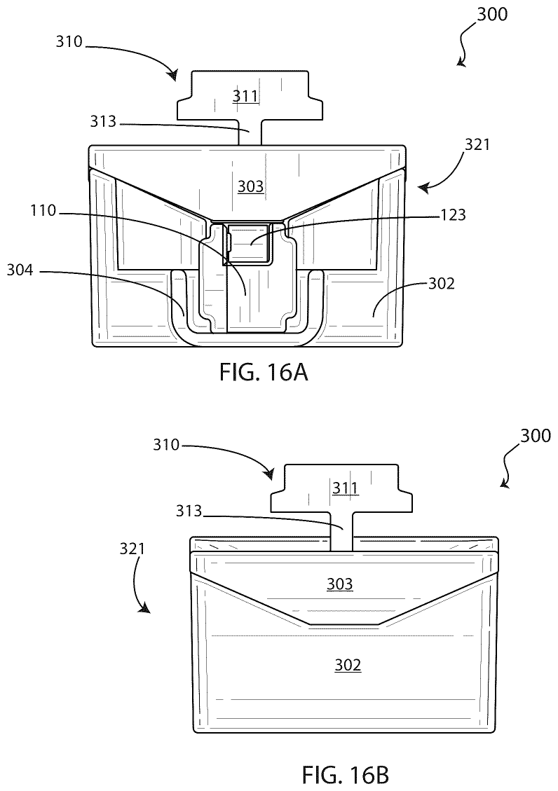

FIG. 16A is a distal end view of a loading tray for a tissue graft delivery device. FIG. 16B is a proximal end view of a loading tray for a tissue graft delivery device. FIGS. 16A-B show loading tray 300 having a tray proximal end 320 and a tray distal end 321. Body 110 of device 100 is loaded into tray 304 of base 302, as shown in FIG. 16A. Handle 311 is coupled to connector 303 of tamp 210, as seen, and compress 322 is obscured by top 303 of loading tray 300. Pusher block 123 can be seen through open tray proximal end 321 through receiver 304 of base 302. In some embodiments, such as the example embodiment shown in FIG. 16B, a tray distal end 321 is closed wherein components of a chute 110 of a tissue graft delivery device inserted into loading tray 300 are covered by base 302 and top 303.

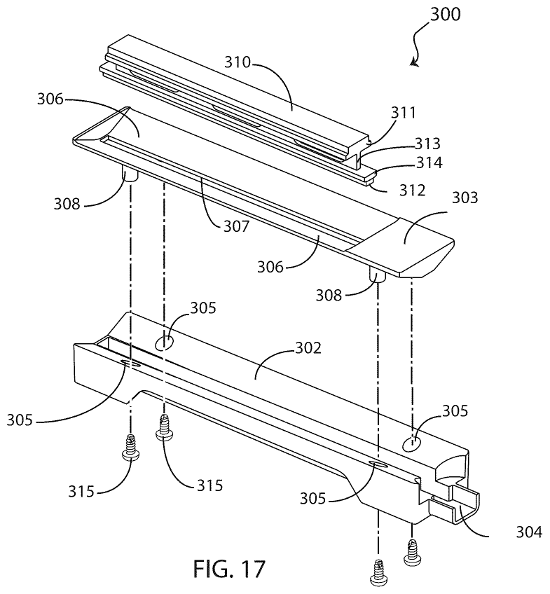

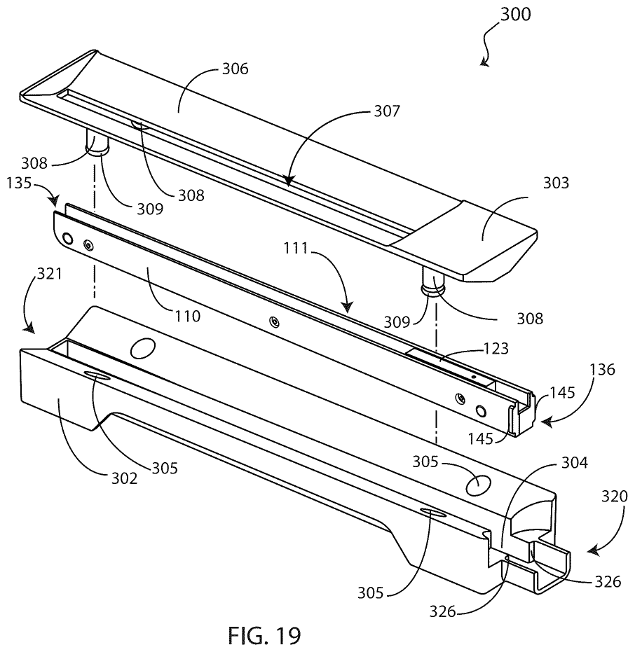

FIG. 17 is an exploded view of a loading tray for a tissue graft delivery device. FIG. 17 shows base 302 having a plurality of base mating features 305 and a corresponding plurality of top mating features 308 coupled to top 303. In some embodiments, each base mating feature 305 reversibly couples to a correspondingly positioned top mating feature 308, such as by interaction of a male shape with a female shape, as shown in FIG. 17 wherein base mating feature 305 is formed as a female shape and top mating feature 308 is formed as a male shape. In some embodiments, a surface feature 309 presented by feature 305 and/or feature 308 interact to reversibly lock feature 305 and feature 308 together (not shown on FIG. 17; see FIG. 19, e.g.). In some embodiments, feature 308 simply slides into feature 308 without locking or additional interaction. In some embodiments, such as the example shown in FIG. 17, a plurality of removable fastening means 315 are used to couple top 303 to base 302 of loading gray 300 passing through base mating feature 305 and top mating feature 308, as shown, prior to loading of a tissue graft into a chute of a tissue graft delivery device. Fastening means 315 may be screws (as shown), other mechanical fasteners such as clips, magnetic fasteners, or other means. In some embodiments of loading tray 300, fastening means are not used.

In some embodiments, a lip 315 disposed between connector 313 and compress 322 is dimensioned to rest on sloping surface 306 to allow passage of compress 322 through opening 307 only to a limited degree. This is shown on FIG. 17, e.g.

In some embodiments of loading tray 300, base 302 and top 303 are formed as a unitary body, wherein a user loads a body with a chute of a tissue graft delivery device by inserting the body into receiver 304 from tray proximal end 320 and advancing the body distally along receiver 204 until the body is fully inserted into loading tray 300, such as shown by body 110 with chute 111 fully inserted into loading tray 300 in FIG. 15A and FIG. 16A and FIG. 18A, for example.

FIG. 18A is a side view of a loading tray for a tissue graft delivery device containing a chute of a tissue graft delivery device. FIG. 18B is a top view of a loading tray for a tissue graft delivery device containing a chute of a tissue graft delivery device. FIG. 18C is a bottom view of a loading tray for a tissue graft delivery device. FIGS. 18A-C show views of a fully assembled loading tray 300. Handle 311 coupled to connector 313 of tamp 310 are seen. Compress 322 rests inside opening 307, which is obscured by lip 314 resting on sloping side 306 of top 303. In some embodiments, such as shown in FIG. 18C, one or more fastening means 315 are inserted through one or more base mating features 305 to couple top 303 to base 302.

FIG. 19 is an exploded view of a loading tray for a tissue graft delivery device, including a chute of a tissue graft delivery device. As shown in FIG. 10, receiver 304 runs axially along base 302 between tray proximal end 320 and tray distal end 321 of loading tray 300. Body 110 fits within receiver 304 for loading of bone graft or other tissue graft material into chute 111 of body 110. In some embodiments, body 110 is placed into receiver 304 from the top of base 302 after which top 303 is then coupled to base 302 over body 110. In some embodiments wherein top 303 is coupled to base 302 prior to inserting body 110 into loading tray 300, a body distal end 221 is positioned within receiver 304 at tray proximal end 320 and thereupon advanced distally through receiver 304 until body 110 is fully inserted into loading tray 300. “Fully inserted,” in this specific context, means that a body proximal end 136 is completely within receiver 304. In some embodiments, a shelf 125 is disposed at body proximal and 136. Shelf 125 is configured to interact with a stop 316 disposed on base 302 such that when body 110 is fully inserted into loading tray 300, stop 316 prevents further distal advancement of body 110 into receiver 304. Additionally, the blocking action of stop 326 against shelf 125 indicates to the user that body 110 has been fully inserted into receiver 304 and is in a proper position for loading of bone graft or other tissue graft material into chute 111.

FIG. 19 additionally shows surface feature 309 of top mating feature 308. In some embodiments, surface feature 309 is configured to “snap” across a corresponding surface feature of base mating feature 305 whereupon the interaction of the top and base surface features reversibly locks top 303 to base 302. In some embodiments of loading tray 300, such reversibly locking surface features 309 of top 303 and base 302 are present without fastening means 315. In some embodiments, fastening means 315 is present without surface feature 309. In some embodiments, both surface features 309 and fastening means 315 are used together to couple top 303 to base 302.

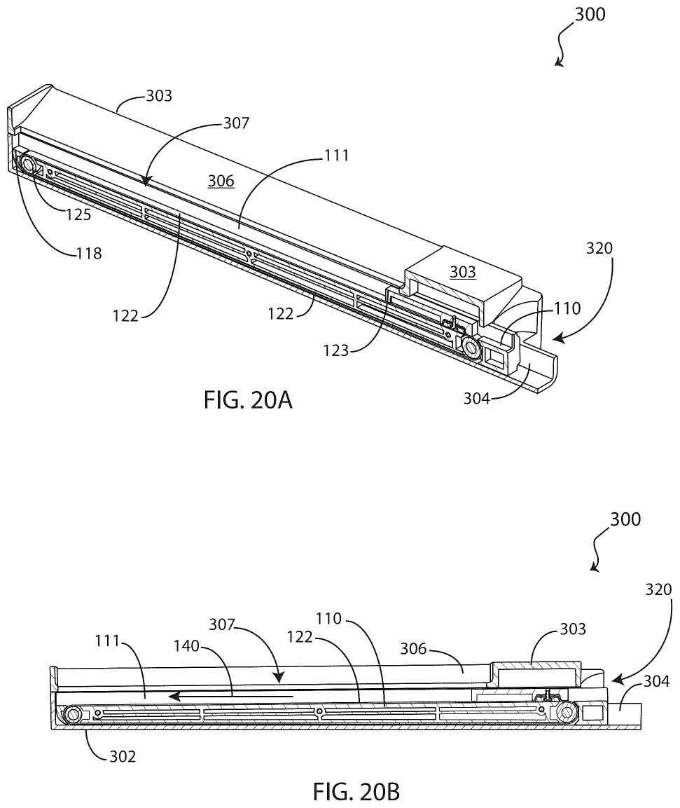

FIG. 20A is a perspective cutaway view of a loading tray for a tissue graft delivery device containing a chute of a tissue graft delivery device, and FIG. 20B is a side cutaway view of a loading tray for a tissue graft delivery device containing a chute of a tissue graft delivery device. FIG. 20A-B show body 110 of tissue graft delivery device 100 fully inserted within receiver 104 of loading tray 300. In the configuration shown in FIGS. 20A-B, chute 111 of device 100 is ready to be loaded with bone graft or other suitable tissue graft material through opening 197 of top 300. For example, in some embodiments, bone graft is placed along sloping side 306 and opening 307 of loading tray 300 wherein graft delivery device 100 has been fully inserted. The bone graft is then moved through opening 307 and gently packed into chute 111. Moving of the graft into chute 111 and gentle packing of the graft into chute 111 is be performed with the aid of a tamp 310, in some embodiments as discussed herein. Following loading of the graft to substantially fill chute 111, body 110 of delivery device 100 is removed from loading tray 300, either by withdrawing body 110 from tray proximal end 320 or decoupling top 303 from base 302, thereupon lifting and removing body 110 from receiver 304 from base 302. Belt movement 140 is also shown in FIG. 20B, in response to an actuator 130, for example.

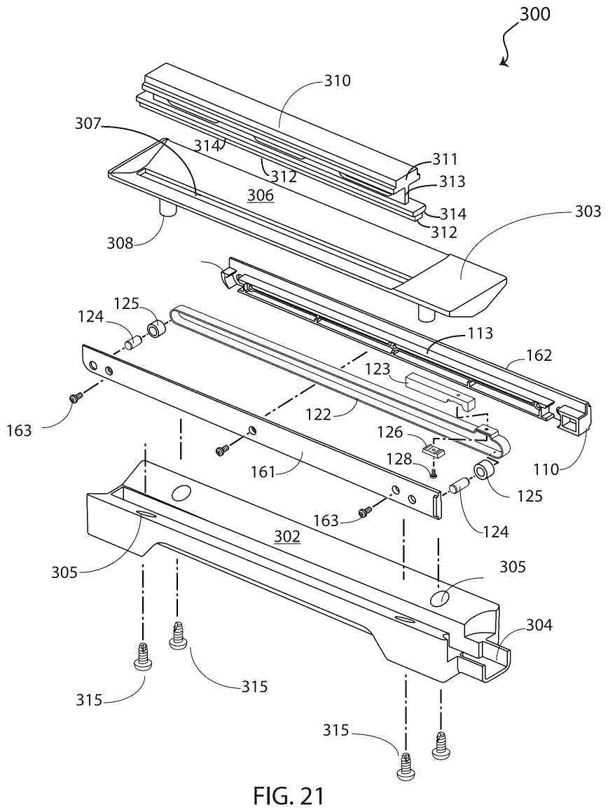

FIG. 21 is an exploded view of a loading tray for a tissue graft delivery device, combined with a exploded view of a chute of a tissue graft delivery device. FIG. 21 shows components of graft delivery device 100 that, when assembled, form chute 111 and contain belt 122 and the several components for mounting and associated with actuating movement of belt 122, in some embodiments and as already discussed at length herein. Notably, FIG. 21 also shows tamp 310 comprising handle 311 and compress 322 coupled by connector 313. In this example and in some embodiments, handle 312, connector 313, and tamp 322 are formed as a unitary body. Methods of formation may include injection molding, 3-D printing, extrusion, or others depending on the type of material (i.e., polymeric plastics, metals, or metal alloys) used to form tamp 322, as known in the art. FIG. 21 also shows lip 314 running along a length and a width of compress 312 dimensioned such that compress 312 is slightly smaller than opening 307 and that lip 314 is larger then opening 307 such that lip 314 limits the depth to which compress 312 may be inserted into chute 111 through opening 307 of a tissue delivery device fully inserted into receiver 304 of loading tray 300.

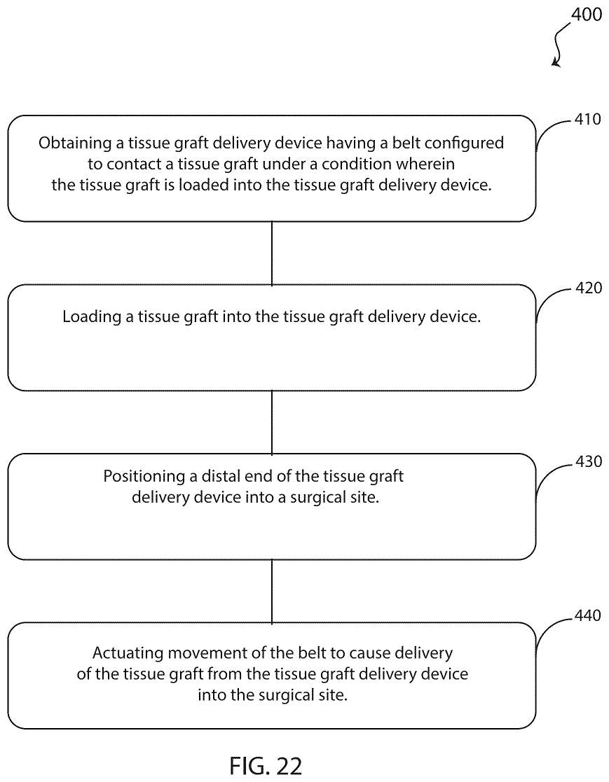

FIG. 22 is a diagrammatic representation of a method of using a tissue graft delivery device. FIG. 22 shows method 400 comprising an obtaining step 410, a loading step 420, a positioning step 430, and an actuating step 440.

Obtaining step 410, in some embodiments, comprises obtaining a tissue graft delivery device having a belt configured to contact a tissue graft under a condition wherein the tissue graft is loaded into the tissue graft delivery device.

Loading step 420, in some embodiments, comprises loading a tissue graft into the tissue graft delivery device.

Positioning step 430, in some embodiments, comprises positioning a distal end of the tissue graft delivery device into a surgical site.

Actuating step 440, in some embodiments, comprises actuating movement of the belt to cause delivery of the tissue graft from the tissue graft delivery device into the surgical site.

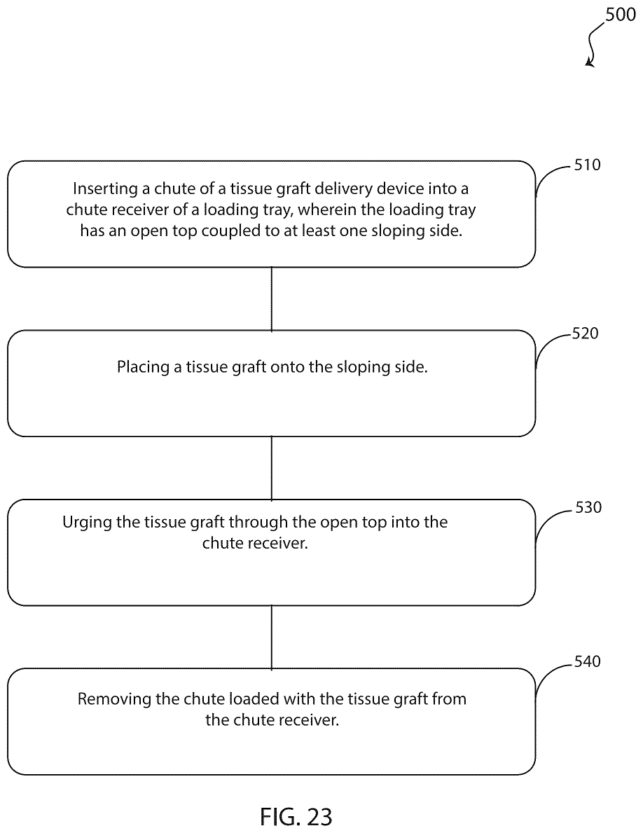

FIG. 23 is a diagrammatic representation of a method of loading a tissue graft delivery device. FIG. 23 shows method 500 comprising an inserting step 510, a placing step 520, an urging step 530, and a removing step 540.

Inserting step 510, in some embodiments, comprises inserting a chute of a tissue graft delivery device into a chute receiver of a loading tray, wherein the loading tray has an open top coupled to at least one sloping side.

Placing step 520, in some embodiments, comprises placing a tissue graft onto the sloping side.

Urging step 530, in some embodiments, comprises urging the tissue graft through the open top into the chute receiver.

Removing step 540, in some embodiments, comprises removing the chute loaded with the tissue graft from the chute receiver.

Components of bone graft delivery device 100, such as sleeve 101 and body 110, may be manufactured from suitable biocompatible materials known in the art for use in forming medical device components designed for surgical procedures. Examples of such materials include thermoplastics such as polycarbonate, polypropylene, polyethylene, or custom-formulated thermoplastic polymers. Metal or metal-alloy components may also be used, such as aluminum, stainless steel, and the like.

The embodiments and examples set forth herein were presented in order to best explain the present invention and its practical application, and to thereby enable those of ordinary skill in the art to make and use the invention. However, those of ordinary skill in the art will recognize that the foregoing description have been presented for the purpose of illustration and example. The description as set forth is not intended to be exhaustive or to limit the invention to the precise device forms and methods disclosed. Many modifications and variations are possible, in light of the teachings herein.

What is claimed is:

1. A tissue graft delivery device (100) comprising:
a body (110) having a distal opening (103); and
a belt (122) mounted to the body (110),
wherein a first friction (151) between a tissue graft contacting the belt and the belt exceeds a second friction (150) between the tissue graft (180) and the body (110) causing the tissue graft (180) to move axially through the body (110) towards the distal opening (103) and to exit the body (110) through the distal opening (103) in response to a movement of the belt (122).

2. The tissue graft delivery device (100) of claim 1, further comprising an actuator (130) functionally coupled to the belt (122), wherein the actuator (130) causes the movement of the belt (122).

3. The tissue graft delivery device (100) of claim 2, wherein the actuator (130) is a rod (131) contacting a pusher block (123) coupled to the belt (122).

4. The tissue graft delivery device (100) of claim 2, wherein the actuator (130) comprises a rack-and-pinion gear mechanism.

5. The tissue graft delivery device (100) of claim 2, wherein the actuator (130) is manually powered.

6. The tissue graft delivery device (100) of claim 2, wherein the actuator (130) is electrically powered.

7. The tissue graft delivery device (100) of claim 1, wherein the tissue graft (180) comprises a bone tissue.

8. The tissue delivery device (100) of claim 1, wherein the body (110) comprises:
a chute (111) having a substantially elongate shape formed by at least one side wall (120) and a floor (113) defining a partially enclosed channel, wherein the belt (122) is disposed within the channel, and
an open top (108) forming a longitudinal opening in the channel opposite the floor (113), and
a sleeve (111) having:
a length (105) equal to or longer than the chute length (112);
a proximal opening (102); and
a distal opening (103),
wherein the sleeve (102) is configured to removably receive the chute (111) and to constrain the tissue graft (180) within the chute (111).

9. The tissue graft delivery device (100) of claim 8, wherein an actuator (130) is movably disposed through the proximal end (116).

10. The tissue delivery device (100) of claim 8, additionally comprising a pusher block (123).

11. The tissue delivery device (100) of claim 8, wherein the pusher block (123) is fixedly coupled to the belt (122) and wherein distal movement of the pusher block (123) within the chute (111) in response to the actuator (130) causes the belt (122) to rotate.

12. A tissue graft delivery device (100) comprising:
a body (110) having:
a substantially elongate shape, at least one side wall (120) defining a chute (111) that is partially enclosed, and a distal opening (103);
a belt (122) mounted within the chute (111), wherein the belt (122) forms a floor (113) of the chute (111);
an open top (108) extending axially along a portion of the length of the chute (111) configured such that a tissue graft (180) is loaded into the chute (111) through the open top 108); and
a sleeve (101) having a first (proximal) opening (102) and configured to receive the body (110), wherein the sleeve (101) constrains the tissue graft (180) loaded into the chute (111); and
wherein the tissue graft (180) is moved out of the tissue graft delivery device (100) through the distal opening (103) in the chute (111) in response to movement of the belt (122).

13. The tissue graft delivery device (100) of claim 12, wherein the belt (122) is positioned opposite the open top (108) and generally parallel to the open top (108).

14. A loading tray for a tissue graft delivery device, comprising
a base having a chute receiver dimensioned to receive a chute assembly of a graft delivery device,
a sloping side positioned proximate to the chute assembly loaded into the receiver and configured to direct a tissue graft placed on the sloping side into the chute positioned within the chute receiver; and
a tamp configured to compress the tissue graft into the chute.

15. The loading tray of claim 14, further comprising a top, wherein the top is elongated, comprises two sloping sides bounding an opening having a length about equal to the length of the chute.

16. The tissue graft delivery device (100) of claim 12, wherein the sleeve (101) is configured to removably receive the body (110).

17. The tissue graft delivery device (100) of claim 12, wherein the chute (111) is configured with an open top (108) that is at least partially covered by the sleeve (101) such that loading of the chute (111) with the tissue graft (108) requires removal of the body (110) from the sleeve (101).

18. The tissue graft delivery device (100) of claim 8, wherein the body (110) is dimensionally configured to slide into the sleeve (101) through a proximal opening (102) of the sleeve (101).

19. The tissue graft delivery device (100) of claim 12, wherein the chute (111) has an open top (108) through which the tissue graft (180) is loaded into the chute (111).

20. The tissue graft delivery device (100) of claim 12, wherein the sleeve (101) comprises a spout (104).
